(19) United States
(12) Patent Application Publication
(10) Pub. No.: US 2025/0171382 A1
(43) Pub. Date: May 29, 2025

(54) SYSTEMS AND PROCESSES FOR CATALYTIC CONVERSION OF C1-C5 ALCOHOLS TO C2-C5 OLEFIN MIXTURES

(71) Applicants: Gevo, Inc., Englewood, CO (US); LG CHEM, LTD., Yeongdeungpo-gu, Seoul (KR)

(72) Inventor: Jonathan Smith, Highlands Ranch, CO (US)

(21) Appl. No.: 18/948,641

(22) Filed: Nov. 15, 2024

Related U.S. Application Data
(60) Provisional application No. 63/603,330, filed on Nov. 28, 2023.
(60) Provisional application No. 63/627,521, filed on Jan. 31, 2024.

(51) Int. Cl. C07C 5/333; C07C 7/00

(52) U.S. Cl. CPC C07C 5/3335; C07C 7/00; C07C 2521/04; C07C 2521/06; C07C 2523/04; C07C 2527/14; C07C 2529/40

(57) ABSTRACT

Processes for converting one or more C₁-C₅ linear or branched alcohols to one or more C₂-C₅ olefins are provided. In one exemplary aspect, the process can be a single stage process for the direct conversion of C₁-C₅ alcohols to olefinic mixtures (e.g., C₂-C₅) carried out in a reactor using a catalyst that includes a zeolite doped with at least one or more metal dopants. Systems for carrying out these processes are also provided.

### CROSS-REFERENCE TO RELATED APPLICATIONS

This application claims priority to U.S. Provisional Patent Application No. 63/627,521 filed on Jan. 31, 2024 and U.S. Provisional Patent Application No. 63/603,330 filed on Nov. 28, 2023, each entitled “SYSTEMS AND PROCESSES FOR CATALYTIC CONVERSION OF C1-C5 ALCOHOLS TO C2-C5 OLEFIN MIXTURES,” the disclosures of which are incorporated herein by reference in their entireties.

### TECHNICAL FIELD

Systems and processes for catalytic conversion of C₁-C₅ alcohols, and more specifically, to catalytic processes resulting in the direct conversion of bio-based C₁-C₅ alcohols to olefinic mixtures (e.g., C₂-C₅) are provided.

### BACKGROUND

There is an increasing demand for the use of biomass for partly replacing petroleum resources for the synthesis of fuels. The use of bioethanol for the synthesis of base stocks for fuels is therefore of great interest. The reaction at the root of the process of converting ethanol to a base stock for fuels is ethanol dehydration followed by ethylene oligomerization.

In most ethanol dehydration processes, ethanol conversion is nearly complete. The increase of C₂-selectivity while keeping high ethanol conversion is of importance to gain in process efficiency and to save expensive steps of downstream separation/purification. It is well known that dehydration occurs readily on acid solids at temperatures above 300° C. The reaction products are mainly water and ethylene, ethylene being obtained with selectivity's as high as 96+%. The most commonly used catalysts are high purity gamma aluminas, silica-aluminas, unprocessed zeolites (e.g. ZSM-5) or zeolites modified by steaming. Additionally, the presence of water in the ethanol feed would also have the effect of limiting catalyst surface deactivation.

The oligomerization of ethylene requires high pressures, generally ranging between 2-4 MPa, but lower temperatures, generally between 20° C.-200° C. The catalysts used are in most cases transition metals deposited on silica-alumina type supports, zeolites (ZSM-5) or mesoporous solids (MCM-41). However, the direct oligomerization of ethylene results in relatively low amounts (˜40% highest reported level) of C₁₀₊ or diesel fraction. Alternatively, the oligomerization of ethylene to C₈₊ olefins can be accomplished via a two-stage process. The first stage encompasses dimerization of a purified ethylene stream to butenes, followed by second stage oligomerization of butenes to C₈₊ olefins which provides the base stock for fuels after hydrogenation.

Accordingly, there remains a needs for improved, efficient, and cost effective catalytic processes resulting in the direct conversion of bio-based alcohols to olefinic mixtures.

### SUMMARY

Aspects of the current subject matter relate inter alia to systems and processes for converting one or more C₁-C₅ linear or branched alcohols to one or more C₂-C₅ olefins.

Exemplary processes for converting one or more C₁-C₅ linear or branched alcohols to one or more C₂-C₅ olefins are disclosed. In one exemplary aspect, the process includes contacting an input stream comprising the one or more C₁-C₅ linear or branched alcohols with a catalyst in a reactor to form an output stream that includes the one or more C₂-C₅ olefins. The catalyst includes a zeolite and one or more metal dopants, where the one or more metal dopants include one or more first metal dopants, one or more second metal dopants, or both. The reactor is at a temperature from about 300° C. to about 600° C., a gauge pressure from 0 to about 30 bar, and a weight hourly space velocity (WHSV) from about 0.25 h⁻¹ to about 35 h⁻¹.

In some aspects, the process can include prior to contacting the input stream, adding the one or more metal dopants to the catalyst.

In some aspects, the reactor can be a single bed reactor. In certain aspects, the single bed reactor can be a fixed bed reactor. In other aspects, the single bed reactor can be a fluidized bed reactor. In yet other aspects, the single bed reactor can be a moving bed reactor.

In some aspects, the one or more C₂-C₅ olefins can be present in the output stream in an amount that does not exceed about 98 wt. % of the total amount of unsaturated hydrocarbons present in the output stream. In certain aspects, the one or more C₂-C₅ olefins can be present in the output stream in an amount that is from about 70 wt. % to about 98 wt. % of the total amount of unsaturated hydrocarbons present in the output stream. In other aspects, the one or more C₂-C₅ olefins can be present in the output stream in an amount that is from about 85 wt. % to about 98 wt. % of the total amount of unsaturated hydrocarbons present in the output stream.

In some aspects, the catalyst can further include one or more non-metal dopants. In certain aspects, the one or more non-metal dopants can include boron, phosphor, or a combination thereof. In one aspect, the boron can be present in the catalyst in an amount from about 0.01 wt. % to about 10 wt. %. In another aspect, the boron can be present in the catalyst in an amount of at least 0.05 wt. %. In one aspect, the phosphor can be present in the catalyst in an amount from about 0.1 wt. % to about 7 wt. %. In another aspect, the phosphor can be present in the catalyst in an amount of at least 1.5 wt. %.

In some aspects, the zeolite can include a ZSM-5 zeolite.

In some aspects, the zeolite can include a MFI zeolite, a BEA zeolite, a FER zeolite, a CHA zeolite, a FAU zeolite, or any combination thereof.

In some aspects, the output stream can include saturates, in which a total amount of saturates can be present in the output stream does not exceed about 25 wt. % of the output stream. In certain aspects, the total amount of saturates present in the output stream can be from about 3 wt. % to 25 about wt. %. In other aspects, the total amount of saturates present in the output stream can be from about 3 wt. % to about 15 wt. %.

In some aspects, the one or more first metal dopants can be present in the catalyst in an amount that does not exceed about 2 wt. %. In certain aspects, the one or more first metal dopants can be present in the catalyst in an amount from about 0.01 wt. % to about 2 wt. %. In other aspects, the one or more first metal dopants can be present in the catalyst in an amount from about 0.01 wt. % to about 0.2 wt. %. In one aspect, the one or more first metal dopants can be present in the catalyst in an amount of at least about 0.05 wt. %

In some aspects, the one or more second metal dopants can be present in the catalyst in an amount that does not exceed about 2 wt. %. In certain aspects, the one or more second metal dopants can be present in the catalyst in an amount from about 0.01 wt. % to about 2 wt. %. In other aspects, the one or more second metal dopants can be present in the catalyst in an amount from about 0.01 wt. % to about 0.2 wt. %. In one aspect, the one or more second metal dopants can be present in the catalyst in an amount of at least about 0.05 wt. %.

In some aspects, the C₁-C₅ linear or branched alcohols can be bio-based and produced by fermentative processes. In other aspects, the C₁-C₅ linear or branched alcohols can be not derived from petroleum.

In some aspects, the process can include removing at least a portion of C₂ olefins from the output stream.

In some aspects, the process can include removing at least a portion of C₄ olefins from the output stream.

In some aspects, the process can include removing at least a portion of C₅ olefins from the output stream.

In some aspects, the temperature can be from about 550° C. to about 600° C. In other aspects, the temperature can be from about 350° C. to about 550° C.

In some aspects, the WHSV can be from about 0.5 h⁻¹ to about 1.0^{h-1}. In other aspects, the WHSV can be from about 2.0 h⁻¹ to about 5.0 h⁻¹.

In some aspects, the one or more first metal dopants can include sodium, potassium, lithium, or any combination thereof. In one aspect, the one or more first metal dopant can be sodium.

In some aspects, the one or more second metal dopant can include magnesium, calcium, strontium, barium, or any combination thereof.

In some aspects, the catalyst can include one or more other dopants. The other dopants can include sulfur, scandium, yttrium, selenium, iron, manganese, tellurium, or any combination thereof. In certain aspects, the one or more other dopants can be present in the catalyst in an amount that does not exceed about 2 wt. %. In other aspects, the one or more other dopants can be present in the catalyst in an amount from about 0.05 wt. % to about 1 wt. %. In one aspect, the one or more other dopants can be present in the catalyst at an amount from about 0.05 wt. % to about 0.5 wt. %. In another aspect, the one or more other dopants can be are present in the catalyst in an amount of at least about 0.25 wt. %

In another exemplary process for converting one or more C₁-C₅ linear or branched alcohols to one or more C₂-C₅ olefins, the process includes contacting an input stream that includes the one or more C₁-C₅ linear or branched alcohols with at least a first catalyst and a second catalyst in a single reactor to form an output stream. The output stream includes the one or more C₂-C₅ olefins. The single reactor is at a temperature from about 350° C. to about 750° C., a gauge pressure from 0 to about 30 bar, and a weight hourly space velocity (WHSV) from about 0.5 h⁻¹ to about 35.0 h⁻¹. The second catalyst includes a zeolite and one or more metal dopants, where the one or more metal dopants include one or more first metal dopants, one or more second metal dopants, or both.

In some aspects, the first catalyst can include a doped or undoped alumina catalyst. In one aspect, the doped alumina catalyst can include, in neutral or ionic form, zirconium, titanium, tungsten, silicon, or any combination thereof.

In some aspects, the single reactor can include one or more catalyst beds, in which the process can include impregnating at least one catalyst bed of the one or more catalysts beds with the first catalyst and the second catalyst. In one aspect, the two of the one or more catalysts beds can be stacked relative to each other within the single reactor.

In some aspects, the single reactor can include two or more catalyst beds, in which the process can include impregnating a first catalyst bed of the two or more catalysts beds with at least the first catalyst, and impregnating a second catalyst bed of the two or more catalysts beds with at least the second catalyst. In one aspect, the two or more catalyst beds can be stacked relative to each other within the single reactor. In certain aspects, contacting an input stream with at least a first catalyst and a second catalyst can include contacting the input stream with the first catalyst bed prior to the second catalyst bed. In one aspect, the process can include introducing one or more additional input streams into the reactor downstream of the first catalyst bed.

In some aspects, the first catalyst bed can be not impregnated with the second catalyst.

In some aspects, the second catalyst bed can be not impregnated with the first catalyst.

In some aspects, the first catalyst can include a silicated γ-alumina, zirconated γ-alumina, titanated γ-alumina, niobium γ-alumina, or fluorinated γ-alumina, an undoped γ-alumina, an undoped zeolite, a silica alumina catalyst, or any combination thereof.

In some aspects, the first catalyst can include an undoped γ-alumina, zirconated gamma-alumina, or both.

In some aspects, the process can include, prior to contacting the input stream, adding the one or more metal dopants to the second catalyst.

In some aspects, the one or more C₂-C₅ olefins can be present in the output stream in an amount that does not exceed about 98 wt. % of the total amount of unsaturated hydrocarbons present in the output stream. In certain aspects, the one or more C₂-C₅ olefins can be present in the output stream in an amount that is from about 70 wt. % to about 98 wt. % of the total amount of unsaturated hydrocarbons present in the output stream. In other aspects, the one or more C₂-C₅ olefins can be present in the output stream in an amount that is from about 85 wt. % to about 98 wt. % of the total amount of unsaturated hydrocarbons present in the output stream.

In some aspects, the second catalyst can include one or more non-metal dopants. The one or more non-metal dopants can include boron, phosphor, or a combination thereof. In certain aspects, boron can be present in the catalyst in an amount from about 0.01 wt. % to about 10 wt. %. In one aspect, boron can be present in the catalyst in an amount of at least 0.05 wt. %. In certain aspects, phosphor can be present in the catalyst in an amount from about 0.1 wt. % to about 7 wt. %. In one aspect, phosphor can be present in the catalyst in an amount of at least 1.5 wt. %.

In some aspects, the zeolite can include a ZSM-5 zeolite.

In some aspects, the zeolite can include a MFI zeolite, a BEA zeolite, a FER zeolite, a CHA zeolite, a FAU zeolite, or any combination thereof.

In some aspects, the output stream can include saturates, in which a total amount of saturates can be present in the output stream does not exceed about 25 wt. % of the output stream. In certain aspects, the total amount of saturates can be present in the output stream is from about 3 wt. % to 25 about wt. %. In other aspects, the total amount of saturates can be present in the output stream is from about 3 wt. % to about 15 wt. %.

In some aspects, the one or more first metal dopants can be present in the second catalyst in an amount that does not exceed about 2 wt. %. In other aspects, the one or more first metal dopants can be present in the second catalyst in an amount from about 0.01 wt. % to about 2 wt. %. In one aspect, the one or more first metal dopants can be present in the second catalyst in an amount from about 0.01 wt. % to about 0.2 wt. %. In another aspect, the one or more first metal dopants can be present in the second catalyst in an amount of at least about 0.05 wt. %.

In some aspects, the one or more second metal dopants can be present in the second catalyst in an amount that does not exceed about 2 wt. %. In other aspects, the one or more second metal dopants can be present in the second catalyst in an amount from about 0.01 wt. % to about 2 wt. %. In one aspect, the one or more second metal dopants can be present in the second catalyst in an amount from about 0.01 wt. % to about 0.2 wt. %. In another aspect, the one or more second metal dopants can be present in the second catalyst in an amount of at least about 0.05 wt. %

In some aspects, the C₁-C₅ linear or branched alcohols can be bio-based and produced by fermentative processes. In other aspects, the C₁-C₅ linear or branched alcohols can be not derived from petroleum.

In some aspects, the process can include removing at least a portion of C₂ olefins from the output stream.

In some aspects, the process can include removing at least a portion of C₄ olefins from the output stream.

In some aspects, the process can include removing at least a portion of C₅ olefins from the output stream.

In some aspects, the temperature can be from about 550° C. to about 600° C. In other aspects, the temperature can be from about 350° C. to about 550° C.

In some aspects, the WHSV can be from about 0.5 h⁻¹ to about 1.0 h⁻¹. In other aspects, the WHSV can be from about 2.0 h⁻¹ to about 5.0 h⁻¹.

In some aspects, the one or more first metal dopants can include sodium, potassium, lithium, or any combination thereof. In one aspect, the one or more first metal dopant can be sodium.

In some aspects, the one or more second metal dopant can include magnesium, calcium, strontium, barium, or any combination thereof.

In some aspects, the second catalyst can include one or more other dopants. The other dopants can include sulfur, scandium, yttrium, selenium, iron, manganese, tellurium, or any combination thereof. In certain aspects, the one or more other dopants can be present in the second catalyst in an amount that does not exceed about 2 wt. %. In other aspects, the one or more other dopants can be present in the second catalyst in an amount from about 0.05 wt. % to about 1 wt. %. In one aspect, the one or more other dopants can be present in the second catalyst at an amount from about 0.05 wt. % to about 0.5 wt. %. In another aspect, the one or more other dopants can be are present in the second catalyst in an amount of at least about 0.25 wt. %.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter of this disclosure are contemplated as being part of the inventive subject matter disclosed herein. It should also be appreciated that terminology explicitly employed herein that also can appear in any disclosure incorporated by reference should be accorded a meaning most consistent with the particular concepts disclosed herein.

### BRIEF DESCRIPTION OF THE DRAWINGS

The accompanying drawings, which are incorporated into and constitute a part of this specification, show certain aspects of the subject matter disclosed herein and, together with the description, help explain some of the principles associated with the disclosed aspects. In the drawings:

FIG. 1 shows an example process concept for an on-purpose propylene configuration of a reactor system with closed-loop recycle of C₂, C₄, and C₅ olefins, consistent with implementations of the current subject matter.

When practical, similar reference numbers denote similar structures, features, or elements.

### DETAILED DESCRIPTION

In the following description, certain specific details are set forth in order to provide a thorough understanding of various aspects. However, one skilled in the art will understand that the disclosure can be practiced without these details. In other instances, well-known structures have not been shown or described in detail to avoid unnecessarily obscuring descriptions of the aspects. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

Reference throughout this specification to “one aspect” or “an aspect” means a particular feature, structure or characteristic described in connection with the aspect is included in at least one aspect. Thus, the appearances of the phrases “in one aspect” or “in an aspect” in various places throughout this specification are not necessarily all referring to the same aspect. Furthermore, the particular features, structures, or characteristics can be combined in any suitable manner in one or more aspects. Also, as used in this specification and the appended claims, the singular forms “a,” “an,” and “the” include plural referents unless the context clearly dictates otherwise. It should also be noted that the term “or” is generally employed in its sense including “and/or” unless the context clearly dictates otherwise.

The word “about” when immediately preceding a numerical value means a range of plus or minus 10% of that value, e.g., “about 50” means 45 to 55, “about 25,000” means 22,500 to 27,500, etc. Furthermore, the phrases “less than about” a value or “greater than about” a value should be understood in view of the definition of the term “about” provided herein.

“Oxygenate” refers to compounds which include oxygen in their chemical structure. Examples of oxygenates include, but are not limited to water, alcohols, esters, and ethers.

“WHSV” refers to weight hourly space velocity and is defined as the weight of the feed flowing per unit weight of the catalyst per hour.

As used herein, “unsaturated hydrocarbons” are organic compounds that are entirely made up of carbon and hydrogen atoms and consist of a double or a triple bond between two adjacent carbon atoms. For example, unsaturated hydrocarbons include olefins, diolefins, alkynes.

“Aromatics” or “aromatic compounds” as used herein refer to any of a large class of unsaturated organic chemical compounds characterized by containing one or more planar rings of carbon atoms joined by covalent bonds of two different kinds (e.g. benzene, naphthalene, etc.).

“Trace amounts” or “trace levels” as used herein refer to levels less than 2%. In some aspects, trace amounts or trace levels can refer to levels less than about 1.5%, less than about 1%, less than about 0.5%, less than about 0.1%, from about 0.1% to about 1.8%, or from about 1% to about 1.5%.

“Single stage transformation” refers to processes which occur within a single reactor system.

“Saturates” as used herein refer to one or more C₂-C₅ paraffins. In some aspects, saturates can include ethane, propane, butanes, pentanes, or any combination thereof.

All yields and conversions described herein are on a weight basis unless specified otherwise.

It is understood that the examples and aspects described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Aspects of the subject matter disclosed herein improve on earlier conversion approaches by, inter alia, providing processes in which Group 1A metal (e.g., sodium, potassium, or lithium) and/or Group 2A metal (e.g., magnesium, calcium, strontium, or barium) doped catalyst system is used to convert C₁-C₅ linear or branched alcohols to C₂-C₇ olefins in high yield with low levels of saturates at competitive costs. Consistent with the current disclosure, processes for the direct conversion of bio-based C₁-C₅ alcohols to olefinic mixtures (e.g., C₂-C₅) with low levels of saturates can be carried out in a single reactor (e.g., using a single catalyst bed or stacked catalyst beds, impregnated with at least one catalyst). The C₂-C₅ olefins can be easily oligomerized to base stocks used in the production of fuels in high yields.

One or more C₁-C₅ linear or branched alcohols can include one or more C₁-C₅ linear alcohols or one or more C₁-C₅ branched alcohols, or both. In some aspects, the one or more linear or branched alcohols include methanol, ethanol, or a combination thereof. In certain aspects, the one or more linear or branched alcohols does not include methanol.

In some aspects, the C₁-C₅ linear or branched alcohols can be bio-based and produced by fermentative processes. In some aspects, the C₁-C₅ linear or branched alcohols are not derived from petroleum.

In some aspects, the processes described herein can be carried out in a single bed reactor (e.g., fixed bed reactor, fluidized bed, or moving bed). In other aspects, the processes described herein can be carried out in a single stacked bed reactor. For example, in certain aspects, alcohols, e.g. methanol or ethanol, can be converted to olefinic mixtures (e.g., C₂-C₅) in a single reactor having a first catalyst in the top section of the reactor with a second catalyst being located in a section of the reactor below the first catalyst. In either the single-stage processes (e.g., using a single bed reactor, e.g., with a mixed catalyst bed) or in the two-stage processes (e.g., using a stacked bed reactor where each catalyst bed is impregnated with at least one catalyst), the resulting C₂-C₅ olefinic mixture is suitable for oligomerization into either gasoline, jet, or diesel fuel cuts at relatively low temperatures and pressures depending upon the oligomerization catalyst selected. Further, in some aspects, the single bed reactor or stacked bed reactor can be defined as a fixed bed reactor, whereas in other aspects, a fluidized bed reactor can be used.

Systems and processes for catalytic conversion of C₁-C₅ alcohols are provided. In general, a catalytic process consistent with the present disclosure includes alcohol dehydration followed by a skeletal carbon build-up and subsequent “cracking” resulting in high yields to low molecular weight olefins (e.g., C₂-C₅). Further, such catalytic processes can result in low levels of saturates (e.g., no greater than 20 wt % of the output stream for a given conversion of alcohol(s) to their corresponding olefins and such corresponding olefins to other hydrocarbons). In other words, for example, the addition of one or more Group 1A metal and/or Group 2A metal dopants to the BP-doped zeolite results in relatively lower levels of saturates as compared to the BP-doped zeolite without the one or more Group 1A metal and/or Group 2A metal dopant) for the same conversion (e.g., 10% versus 15%, respectively, for a complete or near-complete conversion of alcohol(s) to their corresponding olefins and conversion of 60% of such corresponding olefins to other hydrocarbons). In this single stage process, the catalyst mixture can result in a C₂-C₅ olefinic mixture providing access to low molecular weight olefins in yields with good carbon accountability as defined by moles of carbon fed into the system as alcohols versus moles of carbon out of the system incorporated in the C₂-C₅ olefinic mixture. Furthermore, use of recycle streams of specific olefins (e.g., C₂-C₅) advantageously results in the ability to maximize the on-purpose formation of desirable olefins such as propylene, butenes, or mixtures thereof. In some aspects, the mixture of olefins are suitable for oligomerization to either gasoline, jet, or diesel fuel cuts at relatively low temperatures and pressures depending upon the oligomerization catalyst selected.

Typically, most C₂-C₅ alcohols are dehydrated in a single unit operation, at between 300° C.-500° C. in the presence of a dehydration catalyst, resulting in production of the corresponding C₂-C₅ olefin along with water. The water is removed, and the C₂-C₅ olefin is further processed/purified to remove unreacted C₂-C₅ alcohols and/or impurities prior to conversion to chemicals and/or fuels. Relative to ethanol (C₂ alcohol), the classical approach to conversion to chemicals and/or fuels utilizes discrete unit operations to accomplish i) dehydration to ethylene, ii) ethylene purification followed by dimerization to butenes, iii) optionally, olefin metathesis or cracking to propylene, iv) oligomerization of butenes to unsaturated Jet and/or Diesel fuel precursors, or v) direct oligomerization of ethylene to unsaturated Jet and/or Diesel fuel precursors. Similarly, the approach to converting C₃, C₄ or C₅ alcohols to chemicals and/or fuels utilize discrete unit operations to accomplish i) dehydration to the corresponding olefin, ii) olefin purification to remove oxygenates and/or unreacted alcohols, iv) optionally, olefin metathesis or cracking to tailor the distribution of olefins, and/or iii) oligomerization to unsaturated Jet and/or Diesel fuel precursors. Relative to methanol, industrial processes convert methanol, primarily derived from coal, to olefins via a porous catalyst (e.g., SAPO-34, etc.) to olefins in a single step with olefin recycle.

A concept which simultaneously dehydrates, oligomerizes, and cracks C₁-C₅ alcohols or mixtures thereof in one reactor is challenging due to higher temperatures required for complete dehydration (e.g., from about 300° C. to about 500° C.), the large amounts of water present, and the extreme endotherm encountered when C₂₊ alcohols are dehydrated. Implementation of a single unit operation capable of simultaneously dehydrating, oligomerizing, and cracking olefins derived from C₁-C₅ alcohol dehydration requires that catalysts employed be able to withstand high temperatures along with large amounts of water and other oxygenates.

To address these challenges, and to define an approach to convert C₁-C₅ alcohols into a viable feedstock resulting in high yields to fuels, a process has been developed capable of converting C₁-C₅ alcohols, via a single unit operation (e.g., in a single reactor or a single reactor section without intermediate separation), to a mixture of C₂-C₅ olefins in high yield, which is readily separable for use as chemical feedstocks or easily oligomerized to base stocks for fuels in high yield. The ability to accomplish numerous unit operations and chemical transformations in a single reaction process, as presented herein, provides the practitioner with favorable economics due to reduced fixed and variable costs, lower capital investment, less energy, and increased productivity.

To this end, consistent with the current disclosure the conversion of methanol, and/or mixtures of methanol and C₂-C₅ alcohols, proceeds similarly to a C₂-C₅ olefin mixture in high yield and carbon accountability. An exemplary single reaction step encompasses i) dehydration, ii) oligomerization to C₄₊ olefins, iii) skeletal rearrangement, and iv) cracking to predominantly propylene along with C₄₊ olefins and minor amounts of saturates. Thus, passing a vaporized stream of methanol and/or ethanol over a single fixed catalyst bed containing a physical mixture of containing, optionally, a first part of a silicated, zirconated, titanated, niobium, or fluorinated γ-alumina combined with a second part of a doped zeolite (e.g., zeolite doped with boron, phosphor, and a Group 1A metal, e.g., sodium, potassium, or lithium, and/or a Group 2A metal, e.g., magnesium, calcium, strontium, or barium, or a zeolite doped with a Group 1A metal, e.g., sodium, potassium, or lithium) and/or a Group 2A metal, e.g., magnesium, calcium, strontium, or barium at between about 300° C. to about 450° C. results in a C₂-C₅ olefin mixture, which can be separated for sale, or after removal of condensed water, oligomerized “as-is” to primarily jet and/or diesel fuel. This catalyst combination in a single fixed bed reactor accomplishes i) dehydration, ii) oligomerization to C₄₊ olefins, iii) skeletal rearrangement, and iv) cracking that results in longer catalyst time on stream (ToS), improved hydrothermal stability, and improved selectivity to olefins with lesser amounts of saturates and aromatics.

Furthermore, the present systems and processes can optionally include the recycle of one or more specific olefin fractions (e.g., C₂+C₄+C₅ or C₂+C₅, etc.) in a closed-loop process configuration, while co-feeding the C₁-C₅ alcohols. This can result in the maximization of on-purpose yields to selected olefins. For example, the recycle of the C₂+C₄+C₅ olefin fraction in combination with co-feeding C₁-C₅ alcohols using the present system and processes provided herein unpredictably resulted in an on-purpose propylene carbon yield potentially exceeding 80 wt. %. Selective recycle of the C₂+C₅ olefin fraction can result in an on-purpose propylene and butenes combined carbon yield exceeding 80 wt. %. Additionally, recycle of the C₄+C₅ olefin fraction can result in an on-purpose ethylene and propylene combined carbon yield exceeding 80 wt. %. An exemplary single-step reaction can encompass i) in-situ dehydration, ii) oligomerization to C₃₊ olefins, iii) skeletal rearrangement, and iv) cracking to C₂-C₅ olefins along with formation of minor amounts of C₅₊ olefins and aromatics. Recycling the olefin fraction of choice can therefore enable on-purpose olefin production for chemicals and/or fuels production.

Unlike conversion of ethylene, propylene and other olefins of higher molecular weight (C₄₊) can easily be oligomerized over a wide range of catalysts of both zeolitic and non-zeolitic type. The present disclosure, enabling the ability to convert C₁-C₅ alcohols in a single stage, or two-stage reactor configuration in series, to an olefin mixture which includes of primarily C₂-C₅ olefins with low levels of saturates, presents a path towards an economical process to convert C₁-C₅ alcohols to base stocks for chemicals and/or fuels. The process according to the invention implements a scheme that includes a “single” stage transformation of an aqueous C₁-C₅ bio-alcohols feedstock obtained from biomass into primarily C₂-C₅ olefinic mixture, which can be separated to isolate key low molecular weight olefins used throughout the industry as chemical building blocks, or can be easily oligomerized in high yield to C₁₀₊ hydrocarbons or diesel fraction. The two stage or single stage configuration using specific catalytic systems makes it possible to minimize the production of aromatic compounds and therefore maximize production of middle distillates, which constitutes both an asset for the ethanol refiner and an advantage from the standpoint of lasting development.

Conversion of C₁-C₅ alcohols to the desired fuel product, or fuel product precursors (e.g., C₂-C₅ olefins) as in the case of C₁-C₅ alcohols, or mixtures thereof, in a single reactor configuration, can reduce processing costs. In one aspect, a process for converting one or more C₁-C₅ linear or branched alcohols to one or more C₂-C₅ olefins is provided. In some aspects, the process includes a single catalyst system (e.g., a doped zeolite), whereas in other aspects, the process includes a two or more catalyst system (e.g., a dehydration catalyst and a doped zeolite).

In one aspect, the process can include: contacting an input stream that includes one or more C₁-C₅ linear or branched alcohols with a catalyst in a reactor to form an output stream having one or more C₂-C₅ olefins, in which the reactor is at a temperature from about 300° C. to about 600° C., a gauge pressure from 0 to about 30 bar, and a weight hourly space velocity (WHSV) from about 0.25 h⁻¹ to about 35 h⁻¹. The catalyst includes a zeolite and one or more metal dopants, where the one or more metal dopants include one or more first metal dopants, one or more second metal dopants, or both. In certain aspects, the process can further include, prior to contacting the input stream, adding the one or more metal dopants to the catalyst.

The manufacture of zeolite types A, X, and Y is generally carried out by mixing and heating sodium aluminate and sodium silicate solutions, whereupon a sodium aluminosilicate gel is formed. The silicon oxide and aluminum oxide containing compounds pass into the liquid phase from which the zeolites are formed by crystallization. As such, the crude crystalline zeolite containing the original alkali metal may be subsequently converted to an intermediate ammonium form followed by calcination at 500° C.-550° C., to remove the ammonium counterion, thus yielding its' final hydrogen form. In the case of Group 1A metals (e.g., Na, K, Li), the residual alkali metal cation content from the original zeolite synthesis, as described previously, should be in an amount that does not effectively inactivate the catalyst (e.g., less than or equal to 500 ppm). In general, commercially produced hydrogen form zeolites (e.g., Clariant, Zeolyst, etc) typically used for cracking, isomerizations, and alcohol to olefins chemistry have residual sodium levels of less than or equal to 0.05 wt % (e.g., 500 ppm). Higher residual levels of sodium (e.g., >2500 ppm) and other Group 1A metals (e.g., Li, K, etc) can severely deactivate the zeolite catalysts rendering them unacceptable for chemistries requiring highly acidic catalytic activity.

To this end, the inventors were surprised to discover that a subsequent impregnation of a doped zeolite (e.g., doped with boron and phosphor) with low levels of Group 1A metals (e.g., Na<2000 ppm, K<2000 ppm, and/or Li<1000 ppm) results in improved C₁-C₅ alcohol to olefin selectivity as observed by lower levels (˜15%) of aliphatic (e.g., saturates) and aromatic compounds, as compared to a doped zeolite without subsequent Group 1A or Group 1B impregnation. The ability to minimize formation of aliphatic and aromatic compounds, resulting in improved olefin content from alcohols, simplifies downstream separations of the enriched olefin stream to the individual C₂-C₅ olefins or the enriched C₂-C₅ olefin stream be readily oligomerized to fuels in a single unit operation.

Non-limiting examples of suitable zeolites include crystalline silicates of the group ZSM-5 (MFI framework), BEA, CHA, FER, FAU, MWW, MOR, EUO, MFS, ZSM-48, MTT or TON having Si/Al higher than 10, or a dealuminated crystalline silicate of the group ZSM5 (MFI or BEA frameworks), CHA, FER, FAU, MWW, MOR, EUO, MFS, ZSM-48, MTT or TON having Si/Al higher than 10, or molecular sieves of the type silico-aluminophosphate of the group AEL.

A zeolite doped with one or more metal dopants is also referred to herein as a metal doped zeolite. Non-limiting examples of one or more metal dopants (e.g., Group 1A and/or Group 2A metal dopants) include sodium, potassium, lithium, beryllium, magnesium, calcium, strontium, barium, radium, or any combination thereof. In some aspects, the one or more metal dopants include one or more first metal dopants, one or more second metal dopants, or any combination thereof. In certain aspects, the one or more first metal dopants can include a Group 1A metal, such as sodium, lithium, potassium, or any combination thereof, and the one or second metal dopants can include a Group 2A metal, such as magnesium, calcium, strontium, or barium, or any combination thereof.

In some aspects, the zeolite can be doped with only one metal dopant. In one aspect, the zeolite is only doped with sodium. In another aspect, the zeolite is only doped with lithium. In another aspect, the zeolite is only doped with lithium.

In some aspects, the metal doped zeolite can be further doped with one or more non-metal dopants. Non-limiting examples of non-metal dopants include boron, phosphor, or any combination thereof. In one aspect, the one or more non-metal dopants only includes boron and phosphor.

In certain aspects, the catalyst can include a zeolite doped with one or more first metal dopants and one or more non-metal dopants. In some aspects, the catalyst can include a zeolite doped with sodium, lithium, potassium, or any combination thereof and boron, phosphor, or both. In one aspect, the catalyst can include a zeolite doped with sodium, boron, and phosphor. In other aspects, the catalyst can include a zeolite doped with lithium, boron, and phosphor. In yet other aspects, the catalyst can include a zeolite doped with potassium, boron and phosphor. In any of the foregoing aspects, the zeolite can be a ZSM-5 zeolite.

Boron and phosphor can be present in the catalysts in a variety of different amounts. In some aspects, the boron can be present in the catalyst in amount from about 0.01 wt. % to about 10 wt. %, including all the subranges in between. In certain aspects, the boron can be present in the catalyst in an amount from about 0.05 wt. % to about 5 wt. %, including all the subranges in between. In certain aspects, the boron can be present in the catalyst in an amount from about 0.05 wt. % to about 3 wt. %, including all the subranges in between. In one aspect, the boron can be present in the catalyst in an amount of at least 0.05 wt. %. In some aspects, the phosphor can be present in the catalyst in amount from about 0.1 wt. % to about 7 wt. %, including all the subranges in between. In certain aspects, the phosphor can be present in the catalyst in an amount from about 1.5 wt. % to about 6 wt. %, including all the subranges in between. In one aspect, the phosphor can be present in the catalyst in an amount of at least 3 wt. %. In certain aspects, the boron can be present in the catalyst in amount from about 0.5 wt. % to about 3 wt. %, and the phosphor can be present in the catalyst in an amount from about 2 wt. % to 6 wt. %.

In some aspects, the metal doped zeolite can be further doped with additional dopants, also referred to as other dopants, with or without the non-metal dopants. In one aspect, the zeolite is doped with one or more metal dopants, one or more non-metal dopants, and one or more additional dopants. In another aspect, the zeolite is doped with one or more metal dopants and one or more other dopants. Non-limiting examples of additional dopants include iron, tellurium, selenium, cobalt, nickel, lanthanum, chromium, zirconium, ruthenium, molybdenum, iridium, tungsten, copper, manganese, vanadium, zinc, titanium, rhodium, rhenium, gallium, palladium, silver, indium, or any combination thereof.

In another aspect, two or more catalysts can be implemented for the conversion of C₁-C₅ alcohols to the desired fuel product, or fuel product precursors (e.g., C₂-C₅ olefins) as in the case of C₁-C₅ alcohols, or mixtures thereof, in a single reactor configuration. In some aspects, the process includes: contacting an input stream that includes one or more C₁-C₅ linear or branched alcohols with at least a first catalyst and a second catalyst in a single reactor to form an output stream having the one or more C₂-C₅ olefins, in which the single reactor is at a temperature from about 300° C. to about 750° C., a gauge pressure from 0 to about 30 bar, and a weight hourly space velocity (WHSV) from about 0.5 h⁻¹ to about 35.0 h⁻¹. The second catalyst includes a zeolite and one or more metal dopants, in which the one or more metal dopants include one or more first metal dopants, one or more second metal dopants, or both. Furthermore, the two catalysts can be in a stacked bed configuration or admixed together. While a two or more catalyst system is described herein with respect to a single reactor configuration, it is also contemplated herein that such system can be implemented in a two reactor configuration in which at least the first catalyst is in the first reactor and at least the second catalyst in the second reactor.

Exemplary catalyst combinations that can be used in the present two or more catalyst system and processes described herein includes, for example, physically mixed within a single-bed reactor, for C₂-C₅ olefin formation can include one part (e.g., a first catalyst or dehydration catalyst) a silicated, zirconated, titanated, niobium, or fluorinated γ-alumina, an undoped γ-alumina, a zeolite (undoped or doped), a silica alumina catalyst, a solid acid, or any combination thereof. A second part of the catalyst mixture (e.g., a second catalyst) can include a metal doped zeolite. In some aspects, the first catalyst can include undoped gamma-alumina, zirconated γ-alumina, or both, and the second catalyst can include a zeolite doped with sodium, potassium, or lithium, or any combination thereof. In some aspects, the first catalyst can include undoped gamma-alumina, zirconated gamma-alumina, or both, and the second catalyst can include a zeolite doped with magnesium, calcium, strontium, barium, or any combination thereof. In one aspect, the first catalyst can include a doped or undoped alumina catalyst.

Non-limiting examples of suitable zeolites include crystalline silicates of the group ZSM-5 (MFI framework), BEA, CHA, FER, FAU, MWW, MOR, EUO, MFS, ZSM-48, MTT or TON having Si/Al higher than 10, or a dealuminated crystalline silicate of the group ZSM5 (MFI or BEA frameworks), CHA, FER, FAU, MWW, MOR, EUO, MFS, ZSM-48, MTT or TON having Si/Al higher than 10, or molecular sieves of the type silico-aluminophosphate of the group AEL.

Non-limiting examples of one or more metal dopants (e.g., Group 1A and/or Group 2A metal dopants) include sodium, potassium, lithium, beryllium, magnesium, calcium, strontium, barium, radium, or any combination thereof. In some aspects, the one or more metal dopants include one or more first metal dopants, one or more second metal dopants, or any combination thereof. In certain aspects, the one or more first metal dopants can include a Group 1A metal, such as sodium, lithium, potassium, or any combination thereof, and the one or second metal dopants can include a Group 2A metal, such as magnesium, calcium, strontium, or barium, or any combination thereof.

In some aspects, the zeolite can be doped with only one metal dopant. In one aspect, the zeolite is only doped with sodium. In another aspect, the zeolite is only doped with lithium. In another aspect, the zeolite is only doped with lithium.

In certain aspects, the catalyst can include a zeolite doped with one or more metal dopants and one or more non-metal dopants. In some aspects, the catalyst can include a zeolite doped with sodium, lithium, potassium, or any combination thereof and boron, phosphor, or both. In one aspect, the catalyst can include a zeolite doped with sodium, boron, and phosphor. In other aspects, the catalyst can include a zeolite doped with lithium, boron, and phosphor. In yet other aspects, the catalyst can include a zeolite doped with potassium, boron and phosphor. In any of the foregoing aspects, the zeolite can be a ZSM-5 zeolite.

Boron and phosphor can be present in the catalysts in a variety of different amounts. In some aspects, the boron can be present in the catalyst in amount from about 0.01 wt. % to about 10 wt. %, including all the subranges in between. In certain aspects, the boron can be present in the catalyst in an amount from about 0.05 wt. % to about 5 wt. %, including all the subranges in between. In certain aspects, the boron can be present in the catalyst in an amount from about 0.05 wt. % to about 3 wt. %, including all the subranges in between. In one aspect, the boron can be present in the catalyst in an amount of at least 0.05 wt. %. In some aspects, the phosphor can be present in the catalyst in amount from about 0.1 wt. % to about 7 wt. %, including all the subranges in between. In certain aspects, the phosphor can be present in the catalyst in an amount from about 1.5 wt. % to about 6 wt. %, including all the subranges in between. In one aspect, the phosphor can be present in the catalyst in an amount of at least 3 wt. %. In certain aspects, the boron can be present in the catalyst in amount from about 0.5 wt. % to about 3 wt. %, and the phosphor can be present in the catalyst in an amount from about 2 wt. % to 6 wt. %.

In some aspects, the metal doped zeolite can be further doped with additional dopants, also referred to as other dopants, with or without the non-metal dopants. In one aspect, the zeolite is doped with one or more metal dopants, one or more non-metal dopants, and one or more additional dopants. In another aspect, the zeolite is doped with one or more metal dopants and one or more other dopants. Non-limiting examples of additional dopants include iron, tellurium, selenium, cobalt, nickel, lanthanum, chromium, zirconium, ruthenium, molybdenum, iridium, tungsten, copper, manganese, vanadium, zinc, titanium, rhodium, rhenium, gallium, palladium, silver, indium, or any combination thereof.

Granular or extruded catalyst(s) can be used for the reactions described herein. For example, in some aspects, granular or extruded catalyst(s) can have a particle size of greater than at least about 0.05 mm, about 0.1 mm or greater, or from about 0.05 mm to about 2.5 mm, including all the subranges in between. In one aspect, granular or extruded catalysts(s) can have a particle size from about 0.4 to about 2.0 mm.

In certain aspects, the process includes: contacting an input stream that includes the one or more C₁-C₅ linear or branched alcohols with at least a first catalyst and a second catalyst in a single bed reactor to form an output stream that includes the one or more C₂-C₅ olefins, in which the single bed reactor is at a temperature from about 300° C. to about 600° C., a gauge pressure from 0 to about 30 bar, and a weight hourly space velocity (WHSV) from about 0.5 to about 35.0, where the first catalyst includes a doped or undoped alumina catalyst including, in neutral or ionic form, one or more of zirconium (Zr), titanium (Ti), tungsten (W), or silicon (Si); and the second catalyst includes a metal doped zeolite catalyst. Furthermore, the two catalysts can be admixed together in the single bed reactor. We were surprised to find the two catalyst system provides for longer times on stream (ToS) between catalyst regenerations due to the capability of the alumina catalyst component to maintain near quantitative alcohol conversion in comparison to the zeolite component as the alumina component is far less prone to deactivation due to coke formation. In the single catalyst system, unconverted alcohol is detected within 100 h ToS in the reactor condensate effluent in comparison to the two catalyst system which has demonstrated >800 h ToS with trace amounts of ethanol detected in the reactor condensate effluent.

In other aspects, a process for converting one or more C₁-C₅ linear or branched alcohols to one or more C₂-C₅ olefins can include: contacting an input stream that includes the one or more C₁-C₅ linear or branched alcohols with a first catalyst in a stacked bed reactor to form a first mixture that includes the one or more C₂-C₅ olefins. The stacked bed reactor is at a temperature from about 350° C. to about 550° C., a gauge pressure from 0 to about 30 bar, and a weight hourly space velocity (WHSV) from about 1.0 to about 2.0, and the first catalyst includes a doped or undoped alumina catalyst including, in neutral or ionic form, one or more of zirconium (Zr), titanium (Ti), tungsten (W), or silicon (Si). The process further includes contacting the first mixture with at least a second catalyst, where the second catalyst includes a doped metal zeolite catalyst to produce the output stream that includes the one or more C₂-C₅ olefins. In such instances, the first catalyst can be impregnated into a first catalyst bed of the stacked bed reactor, and the second catalyst can be impregnated into a second catalyst bed of the stacked bed reactor

Regarding the output stream, the C₂-C₅ olefins can be present in an amount that is at least 80 wt. % of the total amount of unsaturated hydrocarbons present in the output stream. The C₂-C₅ olefins can be present in an amount from about 70 wt. % to about 99 wt. %, from about 70 wt. % to about 98 wt. %, or from about 85 wt. % to about 98 wt. %, including all the subranges in between, of the total amount of unsaturated hydrocarbons present in the output stream. The C₂-C₅ olefins can be present in an amount that is at least 85 wt. % of the total amount of unsaturated hydrocarbons present in the output stream. The C₂-C₅ olefins can be present in an amount that is at least 90 wt. % of the total amount of unsaturated hydrocarbons present in the output stream. The C₂-C₅ olefins can be present in an amount that is at least 98 wt. % of the total amount of unsaturated hydrocarbons present in the output stream. Further regarding the output stream, the processes disclosed herein can further include removing at least a portion of the C₂ olefins from the output stream. The processes can include removing at least a portion of the C₄ olefins from the output stream. The processes can include removing at least a portion of the C₅ olefins from the output stream.

In some aspects, one or more unsaturated hydrocarbons present in the output stream can include one or more low carbon intensity unsaturated hydrocarbons. In one aspect, all the unsaturated hydrocarbons can be low carbon intensity unsaturated hydrocarbons. As used herein, low carbon intensity when used to modify an unsaturated hydrocarbon (e.g., one or more unsaturated hydrocarbons) refers to a carbon intensity that is at least about 50% less than a typical carbon intensity for its petroleum equivalent.

In some aspects, one or more unsaturated hydrocarbons present in the output stream can include one or more zero carbon intensity hydrocarbons. In one aspect, all the unsaturated hydrocarbons can be zero carbon intensity hydrocarbons. As used herein, zero carbon intensity when used to modify an unsaturated hydrocarbon (e.g., one or more unsaturated hydrocarbons) refers to a carbon intensity that is at least about 90% to 100% less than a typical carbon intensity for its petroleum equivalent.

In some aspects, one or more unsaturated hydrocarbons present in the output stream can include one or more negative carbon intensity hydrocarbons. In one aspect, all the one or more hydrocarbons can be negative carbon intensity hydrocarbons. As used herein, negative carbon intensity when used to modify an unsaturated hydrocarbon refers to a carbon intensity that is more than 100% less than a typical carbon intensity for its petroleum equivalent.

In some aspects, the output stream includes saturates. In certain aspects, a total amount of saturates present in the output stream does exceed about 25 wt. %. In one aspect, a total amount of saturates present in the output stream does not exceed about 20 wt. %. In other aspects, the total amount of saturates present in the output stream can be from about 3 wt. % to 25 about wt. % or from about 3 wt. % to about 15 wt. %, including all subranges in between.

Regarding the reactor, the reactor can be operated at a temperature from about 300° C. to about 600° C., including all the subranges in between. The reactor can be operated at a temperature from about 350° C. to about 550° C., including all the subranges in between. The reactor can be operated at a WHSV from about 0.25 h⁻¹ to about 35.0 h⁻¹, including all the subranges in between. The reactor can be operated at a WHSV from about 0.25 h⁻¹ to 10 h⁻¹ or from about 0.5 h⁻¹ to about 5.0 h⁻¹, from about 5 h⁻¹ to about 10.0 h⁻¹, including all the subranges in between. The reactor can be a fixed bed reactor. The reactor can be a fluidized bed reactor. The reactor can be a moving bed reactor.

The disclosure also describes a process for converting one or more C₁-C₅ linear or branched alcohols to one or more C₂-C₅ olefins using a single catalyst system. In other words, these disclosed processes use a system having only one catalyst. The use of a single catalyst system can be desirable in a variety of instances, for example, when some portion of unconverted C₁-C₅ linear or branched alcohols and related oxygenates are acceptable in the output stream, or when the catalyst is continuously regenerated during operation, which can be implemented in, for example, fluidized bed or moving bed reactors. In some aspects, the one or more C₁-C₅ linear or branched alcohols can be one or more C₁-C₅ linear or branched monohydric alcohols.

Conversion of C₁-C₅ alcohols to the desired fuel product, or fuel product precursors (e.g., C₂-C₅ olefins) as in the case of C₁-C₅ alcohols, or mixtures thereof with a single catalyst system can, for example, reduce processing costs and simplify and optimize the conversion process that would not otherwise be possible with a two-catalyst system. In these processes, the single catalyst system includes only one catalyst, such as zeolite. In some aspects, the only one catalyst is not a doped or undoped alumina catalyst. In certain aspects, the zeolite can be a zeolite doped one or more metal dopants (e.g., Group 1A metal(s) or Group 2A metal(s)), and optionally further doped one or more non-metal dopants (e.g., boron, phosphor, both).

In one aspect, one catalyst can include a zeolite and one or more metal dopants, in which the one or more metal dopants include one or more first metal dopants (e.g., sodium, potassium, lithium, or any combination thereof). In another aspect, the one catalyst can include a zeolite and one or more first metal dopants, in which the one or more first metal dopants include sodium, potassium, lithium, or any combination thereof, and one or more non-metal dopants, in which the one or more non-metal dopants include boron, phosphor, or a combination thereof.

Alternatively, or in addition, the one or more metal dopants can include one or more second metal dopants (e.g., beryllium, magnesium, calcium, strontium, barium, radium, or any combination thereof.) In one aspect, one catalyst can include a zeolite and one or more second metal dopants, in which the one or more second metal dopants include calcium, magnesium, strontium, or any combination thereof. In another aspect, the one catalyst can include a zeolite and one or more second metal dopants, in which the one or more second metal dopants include magnesium, calcium, strontium, or barium, or any combination thereof, and one or more non-metal dopants, in which the one or more non-metal dopants include boron, phosphor, or a combination thereof.

In some aspects, the one or more metal dopants only include one or more Group 1A metals (e.g., one or more first metal dopants). In other aspects, the one or more metal dopants only include one or more Group 2A metals (e.g., one or more second metal dopants). In other aspects, the one or more metal dopants can includes a combination of one or more Group 1A metals and one or more Group 2A metals.

Non-limited examples of zeolites olefin formation can include doped zeolites such as crystalline silicates of the group ZSM-5 (MFI or BEA frameworks), CHA, FER, FAU, MWW, MOR, EUO, MFS, ZSM-48, MTT or TON having Si/Al higher than 10, or a dealuminated crystalline silicate of the group ZSM5 (MFI or BEA frameworks), CHA, FER, FAU, MWW, MOR, EUO, MFS, ZSM-48, MTT or TON having Si/Al higher than 10. In some aspects, when the zeolite is a ZSM-5 zeolite, the ZSM-5 zeolite can have a Si/Al2O3 ratio from about 20 to about 300. In certain aspects, the ZSM-5 zeolite can have a Si/Al2O3 ratio from about 50 to about 150.

In some aspects, the single catalyst system includes only zeolite doped with boron and phosphor, a zeolite doped with boron and phosphor and a Group 1A metal (e.g., sodium, potassium, lithium) and/or a Group 2A metal (e.g., magnesium, calcium, strontium, or barium), or a zeolite doped with a Group 1A metal (e.g., sodium, potassium, lithium) and/or a Group 2A metal (e.g., magnesium, calcium, strontium, or barium). Without being bound by a single theory, it is believed the presence of boron increases the stability of the phosphor within the zeolite framework resulting in extended time on stream (TOS), while also maintaining selectivity. For example, the boron/phosphor doped zeolite maintains >800 h ToS while maintaining good ethylene conversion and olefin selectivity versus <400 h for the phosphor only doped zeolite. Additionally, the presence of boron in such instances can minimize the production of saturates and aromatics in the output stream.

Without being bound by a single theory, it is furthermore believed the presence of Group 1A and/or 2A metal(s) (e.g., at between 0.01 wt. % to 2 wt. %) in combination with boron/phosphor dopants within the zeolite further increases the stability of the boron/phosphor zeolite resulting in extended time on stream (TOS), while also maintaining selectivity due to the additional neutralization of residual strong acid sites within the zeolite framework not modified by the initial boron and/or phosphor impregnation. The ability to effectively titrate residual strong acid sites within the zeolite framework, not initially modified by impregnation with boron and phosphor, improves selectivity as evidenced by a reduction in saturate levels and extended ToS. Furthermore, the strong acid site titration can be accomplished by the simultaneous co-impregnation of boron, phosphor, and the Group 1A and/or Group 2A metal(s). The presence of a Group 1A and/or Group 2A metal(s) in such instances can further minimize the production of saturates and aromatics in the output stream as compared to the boron and phosphor doped zeolite.

The one or more first metal dopants can be present in the catalyst at a variety of different concentrations. In some aspects, the one or more first metal dopants can be present in the catalyst in an amount that does not exceed about 2 wt. %. In other aspects, the one or more first metal dopants can be present in the catalyst in an amount from about 0.01 wt. % to about 2 wt. % or in an amount from about 0.01 wt. % to about 0.2 wt. %. In one aspects, the one or more first metal dopants can be present in the catalyst in an amount of at least about 0.05 wt. %. It is also contemplated that the amount of the one or more first metal dopants presents in the catalyst does not fall outside any of these recited ranges. It is further contemplated that the amount of the one or more first metal dopants presents in the catalyst can be between any of these recited ranges.

The one or more second metal dopants can be present in the catalyst at a variety of different concentrations. In some aspects, the one or more second metal dopants can be present in the catalyst in an amount that does not exceed about 2 wt. %. In other aspects, the one or more second metal dopants can be present in the catalyst in an amount from about 0.01 wt. % to about 2 wt. % or in an amount from about 0.01 wt. % to about 0.2 wt. %. In one aspects, the one or more second metal dopants can be present in the catalyst in an amount of at least about 0.05 wt. %. It is also contemplated that the amount of the one or more second metal dopants presents in the catalyst does not fall outside any of these recited ranges. It is further contemplated that the amount of the one or more second metal dopants presents in the catalyst can be between any of these recited ranges.

In one exemplary aspect, a process for converting one or more C₁-C₅ linear or branched alcohols to one or more C₂-C₅ olefins using a single catalyst system can include contacting an input stream that includes the one or more C₁-C₅ linear or branched alcohols with a catalyst in a reactor to form an output stream that includes the one or more C₂-C₅ olefins, in which the catalyst consists essentially of zeolite doped with boron and phosphor and one or more metal dopants (e.g., Group 1A metals, Group 2A metals, or any combination thereof). The reactor is at a temperature from about 300° C. to about 600° C., a gauge pressure from 0 to about 30 bar, and a weight hourly space velocity (WHSV) from about 0.25 h⁻¹ to about 35 h⁻¹.

In some aspects, the process can include, after contacting the input stream with the catalyst in the reactor, regenerating the catalyst. In some aspects, the regeneration of the catalyst can be carried out by purging any gaseous or liquid hydrocarbons or oxygenates from the reactor and then introducing air and/or oxygen optionally diluted with inert gas or steam to combust any solid carbon deposits on the catalyst. In some aspects, the process can include, a system whereby the catalyst is circulated between a reactor in which it is contacted with the input stream and a regeneration reactor in which is it contacted with air and/or oxygen optionally diluted with inert gas or steam to combust any solid carbon deposits on the catalyst.

In some aspects, the process can further include contacting another input stream that includes the one or more C₁-C₅ linear or branched alcohols with the regenerated catalyst (e.g., the catalyst post-regeneration) in the reactor to form another output stream comprising one or more C₂-C₅ olefins. A person skilled in the art will appreciate that the regenerated catalyst can have a lower concentration of boron, phosphor, or both compared to the catalyst prior to regeneration.

Regarding the reactor, the reactor can be operated at a temperature from about 300° C. to about 600° C., including all the subranges in between. The reactor can be operated at a temperature from about 300° C. to about 550° C., including all the subranges in between. The reactor can be operated at a temperature from about 300° C. to about 500° C. The reactor can be operated at a gauge pressure from 0 to about 30 bar, including all the subranges in between. The reactor can be operated at a gauge pressure from 0 to about 5 bar, including all the subranges in between. The reactor can be operated at a gauge pressure of about 6 or lower. The reactor can be operated at a WHSV from about 0.1 h⁻¹ to about 35 h⁻¹, including all the subranges in between. The reactor can be operated at a WHSV from about 0.25 h⁻¹ to about 10 h⁻¹, including all the subranges in between. The reactor can be operated at a WHSV from about 0.25 to about 5, including all the subranges in between. The reactor can be operated at a WHSV from about 1 to about 10, including all the subranges in between. The reactor can be a fixed bed reactor. The reactor can be a fluidized bed reactor. The reactor can be a moving bed reactor.

Further regarding the output stream, the processes disclosed herein can further include removing at least a portion of the C₂ olefins from the output stream. The processes can include removing at least a portion of the C₃ olefins from the output stream. The processes can include removing at least a portion of the C₄ olefins from the output stream. The processes can include removing at least a portion of the C₅ olefins from the output stream.

In one exemplary aspect, a process for converting one or more C₁-C₅ linear or branched alcohols to one or more C₂-C₅ olefins using a single catalyst system can include contacting an input stream that includes the one or more C₁-C₅ linear or branched alcohols with a catalyst in a reactor to form an output stream that includes the one or more C₂-C₅ olefins. The catalyst consists essentially of a zeolite (e.g., a ZSM-5 zeolite) doped with boron and phosphor and a Group 1A metal (e.g., sodium, lithium, potassium, or any combination thereof). The reactor is at a temperature from about 350° C. to about 475° C., a gauge pressure from 0 to about 5 bar, and a weight hourly space velocity (WHSV) from about 0.25 h⁻¹ to about 10 h⁻¹. The boron is present in the catalyst in an amount from about 0.05 wt. % to about 5 wt. %, the phosphor is present in the catalyst in an amount from about 0.2 wt. % to about 7 wt. %, and the Group 1A metal is present in the catalyst in an amount from 0.01 wt. % to about 2 wt. %.

Group 1A metals, Group 2A metals, or both can be impregnated into a single or respective catalyst bed by any suitable means, for example, via incipient wetness or classical ion exchange methodology.

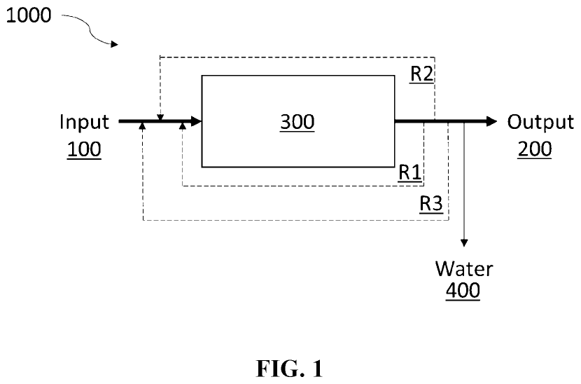

FIG. 1 shows an exemplary reactor system 1000. As shown, an input 100, such as one or more C₁-C₅ linear or branched alcohols can be fed into a reactor 300, to produce an output 200, such as a C₂-C₅ olefin mixture. Additionally, recycle streams R1, R2, and R3 may recycle C₂, C₄, and C₅ olefins respectively, back into the input 100 to be fed back into the reactor 300. Wastewater 400 may also be produced in situ by the fixed bed reactor 300, via dehydration of ethanol to ethylene, and thus condensed and removed as part of the output 200.

Reactor 300 can have a variety of configurations. In some aspects, the reactor is a single bed reactor (e.g., a single fixed bed reactor, single fluidized bed reactor, single moving bed reactor. In such aspects, the single catalyst bed (not shown) of the reactor 300 can be impregnated with two or more catalysts so as to form a mixed catalyst bed.

In other aspects, the reactor 300 can includes two or more catalyst beds (e.g., a stacked configuration). In such aspects, a first catalyst bed can include one or more catalyst impregnated therein and the second catalyst bed can include one or more catalysts impregnated therein. For example, the first catalyst bed can include a first catalyst that includes a silicated γ-alumina, zirconated γ-alumina, titanated γ-alumina, niobium γ-alumina, or fluorinated γ-alumina, an undoped γ-alumina, a zeolite (undoped or doped), a silica alumina catalyst, a solid acid, or any combination thereof; and the second catalyst bed can include a zeolite doped with at least one or more metal dopants (e.g., Group 1A metal(s), Group 2A metal(s), or both). The metal doped zeolite of the second catalyst bed can be further doped with at least one or more non-metal dopants (e.g., boron, phosphor, or both). Alternatively or in addition, the metal doped zeolite can be doped with at least one or more other dopants. In other aspects, the first catalyst bed can include a mixture of the first catalyst and the second catalyst impregnated therein.

In some aspects, the first catalyst bed does not contain the second catalyst. Alternatively, or in addition, the second catalyst bed does not contain the first catalyst.

In aspects where the reactor includes two or more catalyst beds, the reactor can have staged input feeds. In such aspects, for example, the reactor can be a multistage reactor (e.g., adiabatic multistage reactor) having multiple inputs (e.g., at least first and second input feeds), at least a first reaction stage and a second reaction stage, in which the first reaction stage is upstream of the second reaction stage. The first reaction stage includes a first reactor bed and the second reaction stage includes a second reactor bed. In use, a first input feed can be fed into a first end (e.g., an inlet) of the adiabatic multistage reactor and subsequently contact the first reactor bed to produce a first reaction mixture. A second input feed can be introduced into the adiabatic multistage reactor downstream of the first reaction stage, which then mixes with the first reaction mixture to product a first effluent having a different composition relative to the first reaction mixture. The first effluent can then subsequently contact the second reactor bed to product a second reaction mixture. This second reaction mixture can then be pass through the output of reactor, or in other instances to a subsequent reaction stage (e.g., third reaction stage) of the reactor.

In some aspects, during each reaction stage and between reaction stages, external heat is not added to the adiabatic multistage reactor. The phrase “external heat” is heat that is provided to the adiabatic multistage reactor that is not otherwise generated by a chemical reaction within the adiabatic multistage reactor or provided by any input feed (e.g., the first, second, or third input feeds). Alternatively, or in addition, during each reaction stage and between reaction stages, heat is not removed from the adiabatic multistage reactor.

The first input feed can include one or more first oxygenates. For the purposes of this disclosure, an “oxygenate” is a hydrocarbon that contains oxygen as part of its chemical structure. Non-limiting examples of first oxygenates include C₂₊ alcohol(s), such as ethanol, ether(s), ester(s), and the like. In some aspects, the one or more first oxygenates does not include methanol. In some aspects, the one or more first oxygenates can include the same oxygenate, and in other aspects, the one or more first oxygenates can include a mixture of different oxygenates. By way of example, in some aspects, the one or more first oxygenates can include a predominant first oxygenate, for example, ethanol. In such aspects, the one or more first oxygenates can also include one or more other oxygenates, e.g., methanol, propanol, one or more esters and/or one or more ethers. As used herein, a “predominant first oxygenate” can be present at a greater weight percent than any other individual oxygenate in the one or more first oxygenates, for example, present in an amount that is at least 25 weight percent, at least 50 weight percent, or at least 75 weight percent of the one or more first oxygenates. In some aspects, the predominant first oxygenate can be present in an amount of 25 weight percent to 99 weight percent of the one or more first oxygenates, in an amount of 25 weight percent to 90 weight percent of the one or more first oxygenates, in an amount of 50 weight percent to 99 weight percent of the one or more first oxygenates, or in an amount of 75 weight percent to 99 weight percent of the one or more first oxygenates. It is further contemplated that the predominant first oxygenate can be present between any of these recited ranges.

The second input feed includes one or more second oxygenates, so-called as they are the second oxygenates introduced into the reactor at the second stage or catalyst bed. Non-limiting examples of second oxygenates include C₂₊ alcohol(s), such as ethanol, ether(s), ester(s), and the like. In some aspects, the one or more second oxygenates does not include methanol. In some aspects, the one or more second oxygenates can include the same oxygenate, and in other aspects, the one or more second oxygenates can include a mixture of different oxygenates. By way of example, in some aspects, the one or more second oxygenates can include a second predominant oxygenate, for example, ethanol. As used herein, a “predominant second oxygenate” can be present at a greater weight percent than any other individual oxygenate in the one or more second oxygenates, for example, present in an amount that is at least 25 weight percent, at least 50 weight percent, or at least 75 weight percent of the one or more second oxygenates. In some aspects, the predominant second oxygenate can be present in an amount of 25 weight percent to 99 weight percent of the one or more second oxygenates, in an amount of 25 weight percent to 90 weight percent of the one or more second oxygenates, in an amount of 50 weight percent to 99 weight percent of the one or more second oxygenates, or in an amount of 75 weight percent to 99 weight percent of the one or more second oxygenates. It is further contemplated that the predominant second oxygenate can be present between any of these recited ranges.

In other aspects, the reactor system can include two or more reactors in series. In such aspects, for example, when there is a first reactor and a second reactor, the first reactor, the second reactor, or both can have any reactor configuration (e.g., structural design, such as single bed, mixed bed, stacked bed, and the like) disclosed herein. In some aspects, the first reactor and the second reactor have the same configuration (e.g., structural design). In other aspects, the first reactor and the second reactor have different configurations (e.g., structural design). Alternatively, or in addition, the first reactor and the second reactor can operate at the same process conditions (e.g., temperature, pressure, WHSV, and the like). In other aspects, the first reactor and the second reactor can operate at different process conditions. Alternatively, or in addition, the first reactor and the second reactor can each have a catalyst bed and both beds can be impregnated with the same catalyst(s). In other aspects, the catalyst bed(s) of the first reactor can be impregnated with one or more first catalysts and the catalyst bed(s) of the second reactor can be impregnated with one or more second catalyst that are different that the first catalysts.

The following specific examples are intended to be illustrative of the invention and should not be construed as limiting the scope of the invention as defined by appended claims.

### EXAMPLES

### Example 1: Reactor Set-Up

Alcohol (i.e., C₁-C₅) conversion to C₂-C₅ olefins was carried out at 300° C.-500° C., via fixed bed reactors, containing specified catalyst(s), and flowing preheated (160° C.) vaporized alcohol in a downward flow over the fixed catalyst bed while co-feeding nitrogen at atmospheric pressure or under moderate pressures (i.e., 0-30 bar). The flow rate of alcohol was controlled by Teledyne Model 500D syringe pumps, and the flow rates were adjusted to obtain the targeted olefin WHSV (weight hourly space velocity). The internal reaction temperature was maintained constant via a Lindberg Blue M furnace as manufactured by Thermo-Scientific. Alcohol conversion and selectivity was calculated by analysis of the liquid phase reactor effluent by GC for organic and water content, online GC analysis of non-condensed hydrocarbons (i.e., C₂-C₅ olefins), and on-line thermal conductivity detector for quantitation of CO, CO₂ and CH₄ relative to nitrogen as internal standard. Thus, passing a vaporized stream of C₁-C₅ alcohols over the catalyst combination in a single fixed bed reactor at between 350° C.-450° C. results in the formation of C₂-C₅ olefins in high yields.

### Example 2: Impregnated Boron/Phosphor and Sodium (1.5% Boron, 3% Phosphor, 0.13% Sodium) Impregnated ZSM-5 Zeolite Catalyst Preparation

Boron, Phosphor, and Sodium impregnated zeolite catalyst was prepared by incipient wetness technique as described. 0.57 g phosphoric acid (85%), 0.43 g boric acid (99+%), and 0.0238 g sodium nitrate was dissolved in deionized water (3.7 mL). Upon heating and dissolution, the solution was added in dropwise fashion to 5 g ZSM-5 zeolite support (i.e., Zeolyst type CBV-5524 H⁺). The resulting impregnated catalyst was dried at 160° C. for 1 hr, and afterwards calcined at 550° C. for 3-15 hrs.

### Example 3: Single Stage Reactor

Single Stage reactor configuration: Reaction Conditions: T=445° C. in reactor, WHSV=4.9 (ethanol basis), P=0 bar; Catalysts—Siralox 5/320 (5.0 wt % silica) γ-Alumina (0.625 g) physically mixed with B/P/Na (1.5%/3.0%/0.12%) doped ZSM-5 zeolite (2.5 g).

TABLE 1
Single pass reactor effluent composition and
corresponding weight percent of total.

| Single Pass Reactor | |
|---|---|
| Effluent Composition | Wt % of Total: |
| Ethylene | 38.4 |
| Propylene | 18.9 |
| Butenes | 18.1 |
| C₅ olefins | 7.4 |
| C₂—C₅ saturates | 14.2 |
| Aromatics (C₇₊) | <3 |
| C₆₊ olefins | trace |

Ethylene conversion ˜62% mass yield.

### Example 4: Single Stage Reactor

Single Stage reactor configuration: Reaction Conditions: T=445° C. in reactor, WHSV=4.9 (ethanol basis), P=0 bar; Catalysts—High Purity γ-Alumina (Clariant 100-4, 0.625 g) physically mixed with B/P/Na (1.5%/3.0%/0.12%) doped ZSM-5 zeolite (2.5 g).

TABLE 2
Single pass reactor effluent composition and
corresponding weight percent of total.

| Single Pass Reactor | |
|---|---|
| Effluent Composition | Wt % of Total: |
| Ethylene | 33.9 |
| Propylene | 17.9 |
| Butenes | 17.4 |
| C₅ olefins | 7.2 |
| C₂—C₅ saturates | 13.7 |
| Aromatics (C₇₊) | <3 |
| C₆₊ olefins | trace |

Ethylene conversion ˜66% mass yield (MS305).

### Example 5: Single Stage Reactor

Single Stage reactor configuration: Reaction Conditions: T=445° C. in reactor, WHSV=4.9 (ethanol basis), P=0 bar; Catalysts—High Purity γ-Alumina (Clariant 100-4, 0.625 g) physically mixed with B/P/K (1.5%/3.0%/0.18%) doped ZSM-5 zeolite (2.5 g).

TABLE 3
Single pass reactor effluent composition and
corresponding weight percent of total.

| Single Pass Reactor | |
|---|---|
| Effluent Composition | Wt % of Total: |
| Ethylene | 39.8 |
| Propylene | 21.0 |
| Butenes | 17.2 |
| C₅ olefins | 5.2 |
| C₂—C₅ saturates | 13.9 |
| Aromatics (C₇₊) | <3 |
| C₆₊ olefins | trace |

Ethylene conversion ˜60% mass yield.

### Comparative Example 6: Single Stage Reactor

Single Stage reactor configuration: Reaction Conditions: T=445° C. in reactor, WHSV=4.9 (ethanol basis), P=0 bar; Catalysts—Siralox 5/320 (5.0 wt % silica) γ-Alumina (0.625 g) physically mixed with Na doped (0.27 wt %) ZSM-5 zeolite (2.5 g).

TABLE 4
Single pass reactor effluent composition and
corresponding weight percent of total.

| Single Pass Reactor | |
|---|---|
| Effluent Composition | Wt % of Total: |
| Ethylene | 22.6 |
| Propylene | 15.7 |
| Butenes | 16.1 |
| C₅ olefins | 5.9 |
| C₂—C₅ saturates | 26.7 |
| Aromatics (C₇₊) | <13 |
| C₆₊ olefins | trace |

Ethylene conversion ˜77% mass yield.

### Comparative Example 7: Single Stage Reactor

Single Stage reactor configuration: Reaction Conditions: T=445° C. in reactor, WHSV=4.9 (ethanol basis), P=0 bar; Catalysts—High Purity γ-Alumina (Clariant 100-4, 0.625 g) physically mixed with B/P (1.5%/3%) doped ZSM-5 zeolite (2.5 g).

TABLE 5
Single pass reactor effluent composition and
corresponding weight percent of total.

| Single Pass Reactor | |
|---|---|
| Effluent Composition | Wt % of Total: |
| Ethylene | 36.0 |
| Propylene | 18.6 |
| Butenes | 17.7 |
| C₅ olefins | 7.6 |
| C₂—C₅ saturates | 15.0 |
| Aromatics (C₇₊) | <5 |
| C₆₊ olefins | trace |

Ethylene conversion ˜64% mass yield (JS265).

### Example 8: Olefin Selectivity

The data shown in the Table 6 below illustrates improved selectivity propylene with lesser amounts of total C₂-C₅ saturates for a selected time on stream (ToS) timepoint and ethylene conversion for foregoing Examples 4, 5, and 7.

TABLE 6

| | Example | Example | Example |
|---|---|---|---|
| | 4 | 5 | 7 |
| Time on Stream (hr) | 38 | 21 | 40 |
| Pressure (psia) | 15.8 | 14 | 17.9 |
| C2 Conversion | 50 | 50 | 50 |
| (mass yield %) | | | |
| Total Saturates (wt %) | 7 | 6.5 | 8.8 |
| Propylene Purity (%) | 92.1 | 92 | 90 |

Although various illustrative implementations are described above, any of a number of changes can be made to various implementations without departing from the teachings herein. For example, the order in which various described method steps are performed can often be changed in alternative implementations, and in other alternative implementations, one or more method steps can be skipped altogether. Optional features of various system and process implementations can be included in some implementations and not in others. Therefore, the foregoing description is provided primarily for exemplary purposes and should not be interpreted to limit the scope of the claims.

The examples and illustrations included herein show, by way of illustration and not of limitation, specific implementations in which the subject matter can be practiced. As mentioned, other implementations can be utilized and derived there from, such that structural and logical substitutions and changes can be made without departing from the scope of this disclosure. Such implementations of the inventive subject matter can be referred to herein individually or collectively by the term “invention” merely for convenience and without intending to voluntarily limit the scope of this application to any single invention or inventive concept, if more than one is, in fact, disclosed. Thus, although specific implementations have been illustrated and described herein, any arrangement calculated to achieve the same purpose can be substituted for the specific implementations shown. This disclosure is intended to cover any and all adaptations or variations of various implementations. Combinations of the above implementations, and other implementations not specifically described herein, will be apparent to those of skill in the art upon reviewing the above description. Use of the term “based on,” herein and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with implementations related to the described subject matter. Although a few variations have been described in detail herein, other modifications or additions are possible. In particular, further features and/or variations can be provided in addition to those set forth herein. For example, the implementations described herein can be directed to various combinations and subcombinations of the disclosed features and/or combinations and subcombinations of several further features disclosed herein. In addition, the logic flows depicted in the accompanying figures and/or described herein do not necessarily require the particular order shown, or sequential order, to achieve desirable results. Other implementations can be within the scope of the following claims.

1. A process for converting one or more C₁-C₅ linear or branched alcohols to one or more C₂-C₅ olefins, the process comprising:
contacting an input stream comprising the one or more C₁-C₅ linear or branched alcohols with a catalyst in a reactor to form an output stream comprising the one or more C₂-C₅ olefins, the catalyst comprising a zeolite and one or more metal dopants, the one or more metal dopants comprise one or more first metal dopants, one or more second metal dopants, or both;
wherein the reactor is at a temperature from about 300° C. to about 600° C., a gauge pressure from 0 to about 30 bar, and a weight hourly space velocity (WHSV) from about 0.25 h⁻¹ to about 35 h⁻¹.

2. The process of claim 1, further comprising, prior to contacting the input stream, adding the one or more metal dopants to the catalyst.

3.-6. (canceled)

7. The process of claim 1, wherein the one or more C₂-C₅ olefins are present in the output stream in an amount that does not exceed about 98 wt. % of the total amount of unsaturated hydrocarbons present in the output stream.

8.-9. (canceled)

10. The process of claim 1, wherein the catalyst further comprises one or more non-metal dopants.

11. The process of claim 10, wherein the one or more non-metal dopants comprises boron, phosphor, or a combination thereof.

12. The process of claim 11, wherein boron is present in the catalyst in an amount from about 0.01 wt. % to about 10 wt. %, and wherein phosphor is present in the catalyst in an amount from about 0.1 wt. % to about 7 wt. %.

13-21. (canceled)

22. The process of claim 1, wherein the one or more first metal dopants are present in the catalyst in an amount from about 0.01 wt. % to about 2 wt. %.

23.-25. (canceled)

26. The process of claim 1, wherein the one or more second metal dopants are present in the catalyst in an amount from about 0.01 wt. % to about 2 wt. %.

27.-30. (canceled)

31. The process of claim 1, further comprising removing at least a portion of C₂ olefins from the output stream, at least a portion of C₄ olefins from the output stream, removing at least a portion of C₅ olefins from the output stream, or any combination thereof.

32.-37. (canceled)

38. The process of claim 1, wherein the one or more first metal dopants comprise sodium, potassium, lithium, or any combination thereof.

39. (canceled)

40. The process of claim 1, the one or more second metal dopants comprise magnesium, calcium, strontium, barium, or any combination thereof.

41. The process of claim 1, wherein the catalyst comprises one or more other dopants, the one or more other dopants comprising sulfur, scandium, yttrium, selenium, iron, manganese, tellurium, or any combination thereof.

42.-45. (canceled)

46. A process for converting one or more C₁-C₅ linear or branched alcohols to one or more C₂-C₅ olefins, the process comprising:
contacting an input stream comprising the one or more C₁-C₅ linear or branched alcohols with at least a first catalyst and a second catalyst in a single reactor to form an output stream comprising the one or more C₂-C₅ olefins, the single reactor being at a temperature from about 350° C. to about 750° C., a gauge pressure from 0 to about 30 bar, and a weight hourly space velocity (WHSV) from about 0.5 h⁻¹ to about 35.0 h⁻¹,
wherein the second catalyst comprises a zeolite and one or more metal dopants, the one or more metal dopants comprise one or more first metal dopants, one or more second metal dopants, or both.

47. The process of claim 46, wherein the first catalyst comprises a doped or undoped alumina catalyst.

48. (canceled)

49. The process of claim 46, wherein the single reactor comprises one or more catalyst beds, wherein the process further comprises impregnating at least one catalyst bed of the one or more catalysts beds with the first catalyst and the second catalyst.

50. (canceled)

51. The process of claim 46, wherein the single reactor comprises two or more catalyst beds, wherein the process further comprises,
impregnating a first catalyst bed of the two or more catalysts beds with at least the first catalyst, and
impregnating a second catalyst bed of the two or more catalysts beds with at least the second catalyst.

52. (canceled)

53. The process of claim 51, wherein contacting an input stream with at least a first catalyst and a second catalyst comprising contacting the input stream with the first catalyst bed prior to the second catalyst bed.

54. The process of claim 53, further comprising introducing one or more additional input streams into the reactor downstream of the first catalyst bed.

55.-58. (canceled)

59. The process of claim 1, further comprising, prior to contacting the input stream, adding the one or more metal dopants to the second catalyst.

60.-98. (canceled)

99. A process for converting one or more C₁-C₅ linear or branched alcohols to one or more C₂-C₅ olefins, the process comprising:
contacting an input stream comprising the one or more C₁-C₅ linear or branched alcohols with a catalyst in a reactor to form an output stream comprising the one or more C₂-C₅ olefins, the catalyst consisting essentially of a zeolite and one or more metal dopants, the one or more metal dopants comprising one or more first metal dopants, one or more second metal dopants, or both;
wherein the reactor is at a temperature from about 300° C. to about 600° C., a gauge pressure from 0 to about 30 bar, and a weight hourly space velocity (WHSV) from about 0.25 h⁻¹ to about 35 h⁻¹.

100. (canceled)
